(19) United States
(12) Patent Application Publication
(10) Pub. No.: US 2012/0238562 A1
(43) Pub. Date: Sep. 20, 2012

(54) TREATMENT OF CANCER BY TARGETING MOLECULES THAT INFLUENCE MST1/STK4 SIGNALING

(75) Inventors: Bekir Cinar, Los Angeles, CA (US); Filiz Kisaayak Collak, Los Angeles, CA (US)

(73) Assignee: CEDARS-SINAI MEDICAL CENTER, Los Angeles, CA (US)

(21) Appl. No.: 13/415,711

(22) Filed: Mar. 8, 2012

Related U.S. Application Data
(60) Provisional application No. 61/451,034, filed on Mar. 9, 2011.

(51) Int. Cl. A61K 31/5377; A61K 31/4745; A61K 35/00; A61K 31/439

(52) U.S. Cl. 514/232.5; 514/291; 514/293; 514/233.5

(57) ABSTRACT

The invention relates to the treatment of prostate cancer. In various embodiments, the invention teaches a method of administering one or more compounds that inhibit a molecule that antagonizes the activity of tumor suppressor Mst1 and Mst2 pathway signaling. In certain embodiments, one or more of the compounds include an mTOR and PI3K inhibitor.

### CROSS-REFERENCE TO RELATED APPLICATIONS

This application claims priority from U.S. Provisional Patent Application No. 61/451,034, filed on Mar. 9, 2011, which is incorporated herein by reference in its entirety.

### FIELD OF INVENTION

This invention generally relates to cancer prevention and treatment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The Mst1/2 protein kinases (hippo in Drosophila), a component of the RASSF-LATS tumor suppressor network, have been suggested to regulate developmental and carcinogenesis processes in the mammalian system.

There is a need in the art to elucidate the molecular mechanism underlying the regulation of Mst1/2 function in prostate cancer cells, and to develop effective therapeutic strategies based upon that mechanism.

### SUMMARY OF THE INVENTION

In some embodiments, the invention teaches a method of preventing cancer in an individual, including: providing one or more compositions that directly or indirectly inhibit mTOR activity and PI3K activity; and administering a therapeutically effective amount of one or more of the compositions to the individual so as to prevent cancer in the individual.

In some embodiments, the invention teaches a method of inhibiting cancer in an individual, including: providing one or more compositions that directly or indirectly inhibit mTOR activity and PI3K activity; and administering a therapeutically effective amount of one or more of the compositions to the individual so as to inhibit cancer in the individual.

In some embodiments, the invention teaches a method of treating cancer in an individual, including: providing one or more compositions that directly or indirectly inhibit mTOR activity and PI3K activity; and administering a therapeutically effective amount of one or more of the compositions to the individual so as to treat cancer in the individual.

In some embodiments, the invention teaches a method of reducing a rate of cancer tumor development and/or progression to a metastatic state in an individual, including: providing one or more compositions that directly or indirectly inhibit mTOR activity and PI3K activity; and administering a therapeutically effective amount of one or more of the compositions to the individual, so as to reduce the rate of cancer tumor development and/or progression to the metastatic state in the individual.

In certain embodiments, one or more of the compositions reduces a level of Mst1-T120 phosphorylation in an individual with cancer. In certain embodiments, one or more of the compositions includes LY294002 and/or Ku0063794. In certain embodiments, one or more of the compositions includes BEZ-235. In certain embodiments, one or more of the compositions includes rapamycin and/or rapalogs. In some embodiments, the cancer is prostate cancer. In some embodiments, the cancer is hormone refractory metastatic prostate cancer.

In some embodiments, the invention teaches a kit for treating, inhibiting or preventing a cancer in a subject in need thereof, including: providing one or more compositions that directly or indirectly inhibit mTOR activity and PI3K activity; and instructions for the use of the one or more compositions for treating, preventing or inhibiting the cancer in the individual. In some embodiments, the cancer is prostate cancer. In some embodiments, one or more of the compositions reduces a level of Mst1-T120 phosphorylation when administered to an individual with cancer. In some embodiments, one or more of the compositions includes LY294002 and/or Ku0063794. In some embodiments, one or more of the compositions includes BEZ-235.

### BRIEF DESCRIPTION OF THE DRAWINGS

Exemplary embodiments are illustrated in the referenced figures. It is intended that the embodiments and figures disclosed herein are to be considered illustrative rather than restrictive.

FIG. 1 demonstrates, in accordance with an embodiment of the invention, subcellular expression of Mst1-T120 phosphorylation. A) Western blot of Mst1-T120 phosphorylation in the Mcy-tagged antibody immune complex in total lysates from HEK-293 cells transiently transfected with vector (V), Mcy-Mst1-wt or Myc-Mst1-T120A mutant construct. Western blot was performed with pMst1-120 or Myc antibody. B) Western blot of endogenous total or phospho-Mst1-T120 protein in cytoplasmic (Cyt) and nuclear (Nuc) fractions from LNCaP cells that were grown in serum-fed conditions. Equal amounts of cytoplasmic and nuclear protein were loaded on SDS-PAGE in all cell fractionation experiments. C) RNAi knockdown experiment. Cells were transfected with scramble or Mst1 specific siRNA, and levels of total or pMst1-T120 were analyzed in cytoplasmic (C) and nuclear (N) fractions. Lamin A/C was used as a nuclear fraction control in “B” and “C”. D) Immunoprecipitation (IP) with pMst1-T120 antibody. Phospho-Mst1-T120 protein was precipitated from nuclear and cytoplasmic fractions, respectively, and western blots were performed with antibody to the total Mst1 protein. E) Immunofluorescence (IF) staining of pMst-T120 in PCa cells. Alexa Fluor® 488 (green) stained pMst1-T120 protein, and DAPI (blue) stained the nuclei. Magnification was 20×. F) Immunohistochemical (IHC) analysis of clinical samples from non-cancerous or normal prostate (n=5) and cancerous prostate tissues (n=15). Magnification was 20×. IHC was performed using pMst1-T120 and pMst1-T183 antibodies. Images are representative of multiple staining NP: normal prostate; CaP: carcinoma of the prostate. Data are representative of multiple experiments.

FIG. 2 demonstrates, in accordance with an embodiment of the invention, effects of mTOR inhibition alone or together with PI3K on Mst-T120 phosphorylation. A) Schematic representation of the GST-Mst1 peptide with wild type (wt) T120 or T120A mutations. B) In vitro kinase assay with bacterially expressed and purified GST-only, GST-Mst1-T120 wt, or GST-Mst1-T120A mutant peptide and the recombinant pre-activated Akt kinase. Western blots were probed with pMst1-T120 antibody. Ponceau S stained purified GST, GST-Mst1-T120, or GST-Mst1-T120A mutant peptide. C) LNCaP cells were treated with DMSO (vehicle) control, a specific PI3K inhibitor, LY294002 (20 μM), or mTOR inhibitor, Ku0063794 (1 μM) in serum-starved conditions. D) C4-2 cells were treated with Temsirolimus (CCI-779), an mTORC1 inhibitor (1 μM) in addition to DMSO, LY294002 and Ku0063794 under the same experimental conditions as in “C”. E) C4-2 cells treated with DMSO or LY294002 and CCI-779. Control/DMSO or drug treatment in “C,” “D,” or “E” was performed under serum-deprived conditions. Western blots were probed with antibodies to corresponding proteins at 3 h post treatment. Lam-A/C:Lamin-A/C. Data are representative of multiple experiments.

FIG. 3 demonstrates, in accordance with an embodiment of the invention, effects of mTOR inhibition alone or together with PI3K on LNCaP or C4-2 prostate cancer cell proliferation. A, B) LNCaP (A) or C4-2 (B) cells were treated with control (DMSO), LY294002 (20 μM), Ku0063794 (1 μM), rapamycin (1 μM), LY294002+Ku0063794, or LY294002+Rapamycin. C) C4-2 cells were treated with DMSO or the dual PI3K and mTOR inhibitor, BEZ-235 (0.5 μM). Control or drug treatments were performed in T-medium supplemented with 5% FBS and 1% Pen/Strep. Cell proliferation was determined by MTS assay (A490 nm) at 48 h post treatment. D) Levels of endogenous total Mst1 by western blot in cytoplasmic (C) and nuclear (N) fractions from C4-2 cells that were transfected with scramble or Mst1 specific SiRNa. B-actin was used as a loading control. E) Effects of DMSO or CCI-779 on C4-2 cell proliferation with scramble or MSt1 knockdown conditions. Cell proliferation was determined by MTS (A490 nm) at 72 h post transfection. Data are representative of multiple experiments.

FIG. 4 demonstrates, in accordance with an embodiment of the invention, the effects of phosphorylation-deficient (T120A) Mst1 expression on C4-2 cell growth under varying conditions in vitro. A) the blot shows the analysis of ectopically expressed HA-tagged Mst1-wt or MSt1-T120A mutant protein by western blot. Total protein was prepared from TetON-C4-2/Mst1-wt or -C4-2/Mst1-T120A cells. The C4-2/Vector cell model was used as a negative control. B) Clonogenic ability of C4-2 cells expressing stable vector, Mst1-wt, or Mst1-T120A under Dox (0.5 μg/ml) in culture. Colonies were fixed and visualized by crystal violet staining at 7 days post Mst1-wt and Mst1-T120A mutant induction. Graph is the quantification of clones. C) Colony formation of C4-2/vector, C4-2/Mst1-wt, or C4-2/Mst1-T120A cells in soft agar in the presence of Dox (0.5 μg.ml). Graph is the quantification of colonies formed in soft agar. D) 3-dimensional cell growth (sphere formation) in Matrigel. Equal numbers of C4-2/vector, Mst1-wt, or Mst1-T120A cells were seeded. The cells were grown for 10 days in the presence of Dox (0.5 μg/ml). The graph is the quantification of colonies. Engineered C4-2 cells in all experiments were grown in serum-fed conditions. Data are representative of multiple experiments.

FIG. 5 demonstrates, in accordance with an embodiment of the invention, Mst1-T120 phosphorylation negatively regulates the effects of Mst1 on androgen receptor in prostate cancer cells. A) Tumor formation in xenografts. C4-2/vector, C4-2/HA-Mst1-wt, or C4-2/HA-Mst1-T120A cells were subcutaneously inoculated into intact nude male mice. Animals were treated with Dox (0.5 mg/ml) in drinking water for six weeks. Tumor sizes were measured weekly. Tumor volumes were presented as a function of time for each group (n=10). B) PSA promoter reporter activity (p61-LUC) in LNCaP cells that were transfected with Mst1-wt or Mst1-T120A mutant construct, followed by androgen induction in serum-starved conditions. Luciferase reporter assays were performed at 48 h post transfection. The data normalized to the vector control were presented as fold induction with respect to the Mst1-wt. Data are represented as mean +/− SEM. C) Co-immunoprecipitation (co-IP) and western blot (WB) of Mst1 and AR interaction in HEK 293 cells that were transiently transfected with androgen receptor (AR) along with vector, Mcy-Mst1-wt, or Myc-Mst1-T120A mutant. Co-IP with anti-Myc antibody and WB with an antibody to corresponding proteins was performed. Data are representative of multiple experiments. D) Model shows the regulation of the Mst1-T120 phosphorylation by mTOR signaling downstream of the PI3K/Akt pathway in prostate cancer cells. Dotted line represents the indirect regulation of Mst1-T120 by mTOR in cell nuclei.

FIG. 6 demonstrates, in accordance with an embodiment of the invention, A) Mst1 knockdown by gene specific siRNA. LNCaP cells were transfected with scramble or Mst1 specific siRNA and levels of Mst1 were analyzed by western blot in total cell lysates. β-actin was used as loading control. B) Western blots of endogenous total Mst1 or pMst1-T120 protein in cytoplasmic (C) and nuclear (N) fractions of LNCaP cells grown in 10% fetal bovine serum (FBS) or in serum free (SF) conditions. C) Levels of endogenous total or pMst1-T120 in cytoplasmic (C) and nuclear (N) fractions from C4-2B4 or PC-3M prostate cancer cell line and from human embryonic kidney cells (HEK 293) were analyzed by western blot. D) Levels of exogenous Mst1 full-length (FL) and cleaved Mst1-N in cytoplasmic and nuclear fractions from HeLA cells that were transiently transfected with Myc-Mst1-wt or Myc-Mst1-T120A mutant construct. Western blot was performed at 48 h post transfection. Data are representative of multiple experiments.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“AR” as used herein is an acronym for androgen receptor.

“ARE” as used herein is an acronym for androgen-responsive elements.

“GTF” as used herein is an acronym for general transcription factors.

“PI3K” as used herein is an acronym for phosphoinositide-3-kinase.

“mTOR” as used herein is an acronym for mammalian target of rapamycin.

As used herein, “beneficial results” may include, but are in no way limited to, lessening or alleviating the severity of the disease condition, preventing the disease condition from worsening, curing the disease condition, preventing the disease condition from developing, lowering the chances of a subject developing the disease condition and prolonging a subject's life or life expectancy.

“Conditions” and “disease conditions,” as used herein may include, but are in no way limited to cancer, conditions associated therewith or combinations thereof.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

The hippo-like Mst1 serine-threonine kinase has been implicated in poor cancer prognosis in several cancers, including prostate cancer. However, the molecular mechanism of Mst1 regulation in prostate cancer cells remains elusive. Using a custom-designed phospho-Mst1-T120 peptide antibody along with genetic and immunoprecipitation approaches, the inventors demonstrated that Mst1-T120 phosphorylation was enriched in the nucleus, though Mst1 was found in both cell compartments. A similar phosphorylation pattern was also observed in prostate cancer tissue specimens. The inventors' data indicates that attenuation of phosphoinositide-3-kinase (PI3K) or the mammalian target of rapamycin complex 2 (mTORC2) signaling by a potent pharmacologic inhibitor does not significantly alter Mst1-T120 phosphorylation in LNCaP prostate cancer cells. Ironically, inhibition of mTORC1 signaling by a rapamycin analog resulted in Mst1-T120 hyper-phosphorylation in castration-resistant C4-2 cells, but not in the castration-sensitive parental LNCaP line. Combinatorial PI3K and mTOR inhibition significantly reduced Mst1-T120 phosphorylation compared to either single agent. Additional data suggest that sustained T120 phosphorylation is associated with resistance to mTOR inhibition and has a negative impact on Mst1 mediation of growth suppression and inhibition of AR transcriptional activity. These findings reveal a novel mechanism of the Mst/Hippo regulation by mTOR signaling, which has important therapeutic implications in prostate cancer.

In various embodiments, the present invention teaches a method of reducing a rate of cancer tumor development and/or progression to a metastatic state in an individual, including: providing one or more compositions that directly or indirectly inhibit mTOR activity and PI3K activity and/or reduce the level of Mst1-T120 phosphorylation in the individual; and administering a therapeutically effective amount of one or more of the compositions to the individual so as to reduce the rate of cancer tumor development and/or progression to the metastatic state in the individual. In some embodiments, one or more of the compositions inhibits a molecule that antagonizes the activity of tumor suppressor Mst1 and/or Mst2 pathway signaling. In some embodiments, one or more of the compositions includes LY294002 and Ku0063794. In some embodiments, one or more of the compositions includes BEZ-235. In some embodiments, one or more of the compositions includes rapamycin. In some embodiments, the cancer is prostate cancer. In some embodiments, the prostate cancer is characterized in part by an elevated level of Mst1-T120 phosphorylation. In various embodiments, the invention teaches the use of one or more compositions with substantially similar effects as LY294002, Ku0063794, BEZ-235, or rapamycin with respect to inhibiting mTOR or PI3K activity and/or reducing the level of Mst1-T120 phosphorylation in the individual. In some embodiments, the invention teaches the use of one or more rapalogs in a composition of the inventive method. In some embodiments, the individual is a mammal. In some embodiments, the individual is a human.

In some embodiments, the invention teaches a method of preventing, treating, or inhibiting cancer in an individual, including: providing one or more compositions that directly or indirectly inhibit mTOR activity and PI3K activity and/or reduce the level of Mst1-T120 phosphorylation in the individual; and administering a therapeutically effective amount of one or more of the compositions to the individual so as to prevent, treat, or inhibit cancer in the individual. In some embodiments, one or more of the compositions inhibits a molecule that antagonizes the activity of tumor suppressor Mst1 and/or Mst2 pathway signaling. In some embodiments, one or more of the compositions includes LY294002 and Ku0063794. In some embodiments, one or more of the compositions includes BEZ-235. In some embodiments, one or more of the compositions includes rapamycin. In some embodiments, the cancer is prostate cancer. In some embodiments, the prostate cancer is characterized in part by an elevated level of Mst1-T120 phosphorylation. In various embodiments, the invention teaches the use of one or more compositions with substantially similar effects as LY294002, Ku0063794, BEZ-235, or rapamycin with respect to inhibiting mTOR or PI3K activity and/or reducing the level of Mst1 -T120 phosphorylation in the individual. In some embodiments, the invention teaches the use of one or more rapalogs in a composition of the inventive method. In some embodiments, the individual is a mammal. In some embodiments, the individual is a human.

The pharmaceutical compositions according to the methods and kits of the invention may be formulated for delivery via any route of administration. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

The pharmaceutical compositions according to the methods and kits of the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the methods and kits of the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, an elixir, an emulsion or an aqueous or non-aqueous suspension. Such a liquid formulation may be administered directly p.o. or filled into a soft gelatin capsule.

The pharmaceutical compositions according to the methods and kits of the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Typical dosages can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on in vitro responsiveness or the responses observed in the appropriate animal models.

In some embodiments, the present invention is also directed to a kit to treat cancer. In some embodiments, the kit is useful for treating prostate cancer. The kit is an assemblage of materials or components, including one or more of the compositions described herein. Thus, in some embodiments the kit contains a composition that inhibits a molecule that antagonizes the activity of tumor suppressor Mst1 and Mst2 pathway signaling. In some embodiments, one or more of the compositions reduces the level of Mst1-T120 phosphorylation when administered to an individual with cancer. In certain embodiments, one or more of the compositions includes an mTOR and PI3K inhibitor. In some embodiments, one or more of the compositions includes LY294002 and Ku0063794. In some embodiments, one or more of the compositions includes BEZ-235. In some embodiments, one or more of the compositions include rapamycin. In various embodiments, the kit includes one or more compositions with substantially similar effects as LY294002, Ku0063794, BEZ-235, or rapamycin with respect to inhibiting mTOR or PI3K activity and/or reducing the level of Mst1-T120 phosphorylation in the individual. In some embodiments, the invention teaches the inclusion of one or more rapalogs in the inventive kit.

The exact nature of the components configured in the inventive kit depends on its intended purpose. For example, some embodiments are configured for the purpose of treating prostate cancer. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat prostate cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in treating prostate cancer. As used herein, the term “package” refers to a suitable solid matrix or material such as glass, plastic, paper, foil, and the like, capable of holding the individual kit components. Thus, for example, a package can be a glass vial used to contain suitable quantities of an inventive composition that inhibits a molecule that antagonizes the activity of tumor suppressor Mst1 and Mst2 pathway signaling. In some embodiments, one or more compositions are included that reduce the levels of Mst1-T120 phosphorylation when administered to an individual with cancer. In certain embodiments, the composition includes an mTOR and PI3K inhibitor. In some embodiments, the composition includes LY294002 and Ku0063794. In some embodiments, the composition includes BEZ-235. In some embodiments, the composition includes rapamycin. In certain embodiments, the kit comprises one or more compositions with substantially similar effects as LY294002, Ku0063794, BEZ-235, or rapamycin with respect to inhibiting mTOR or PI3K and/or reducing the level of Mst1-T120 phosphorylation when administered to an individual with cancer. In some embodiments, the invention teaches the use of one or more rapalogs in the composition of the inventive kit.

The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described.

The following examples are for illustrative purposes only and are not intended to limit the scope of the disclosure or its various embodiments in any way.

### EXAMPLES

### Example 1

### Introduction

As described herein, the inventors characterized the regulation of Mst1-T120 phosphorylation and its biological significance in prostate cancer cells. The inventors demonstrated that Mst1-T120 phosphorylation was almost exclusively enriched in cell nuclei. The inventors' data indicate that phosphoinositide-3- kinase (PI3K) and mammalian target of rapamycin complex 1 and 2 (mTORC1/2) pathway signaling distinctly regulates Mst1-T120 phosphorylation. The inventors' data also indicate that sustained T120 phosphorylation was associated with resistance to mTOR inhibition and significantly reduced Mst1-induced growth suppression and AR inhibition. These findings demonstrate for the first time that Mst/Hippo and mTOR functionally intersect; a finding with important therapeutic implications in prostate cancer.

### Example 2

### Plasmids, Antibodies and Reagents

Constructions of Myc-tagged and tetracycline-inducible HA-tagged Mst1-wt were described previously (11). The expression of each protein was under the control of the CMV promoter. Phosphorylation-deficient T120A or T387A point mutation on HA-tagged or Myctagged Mst1-wt was generated using a QuickChange site-directed mutagenesis kit (Stratagene, La Jolla, Calif.). Double-stranded oligonucleotide was ligated into the BamH1 and EcoRI sites in pGEX-2TK vector to generate GST-Mst1-T120 fusion. DNA sequencing and enzyme digestions were conducted to verify the orientation and fidelity of all vector constructs. A site-specific phospho-T120 specific Mst1 antibody (pMst1-T120) was custom-made using Mst1 peptide surrounding pT120 as an antigen (GenScript, Inc., Piscataway, N.J.). Other antibodies and reagents used in this study are listed in Example 14 of the present application.

### Example 3

### Cell Fractionations and Protein Analysis

A nuclear extraction kit according to the manufacturer's protocol (Affymetrix, Santa Clara, Calif.) was used to isolate cytoplasmic and nuclear fractions. Total cell lysates were prepared on ice-cold lysis buffer (20 mM HEPES, pH 7.4, 150 mM NaCl, 0.5% NP-40, 1 mM EDTA, protease inhibitors and phosphatase inhibitors). Protein concentrations were determined by the Lowry method (Bio-Rad, Hercules, Calif.). For immunoprecipitation (IP), cleared lysates were incubated with antibody overnight at 4° C. GST-only or GST-Mst1 fusion peptide was expressed in BL21 bacteria (Invitrogen, Inc., Grand Island, N.Y.) with isopropyl-β-D-thiogalactopyranoside (IPTG; 0.75 mM) induction for 4-5 h. Pellets were lysed in buffer 0.1% NP-40, 20 mM Tris-HCl (pH 8.0), 100 mM NaCl, and 1 mM EDTA containing protease and phosphatase inhibitors. Bacterially expressed GST peptides were purified by affinity chromatography on Glutathionesepharose beads (GE Healthcare, Piscataway, N.J.) and stored in PBS at 4° C. Antibody-antigen complexes were collected using Protein A- or G-sepharose (GE Healthcare) and washed three times with lysis buffer. The precipitates were resolved by SDS-PAGE, transferred to nitrocellulose membranes and blocked either with PBST or TBST (0.1% Tween-20) containing 5% (w/v) skim milk powder. Signals were detected using SuperSignal West Pico Chemiluminescence Substrate (Thermo Scientific, Roxford, Ill.).

### Example 4

### Cell Growth Assays

Cell proliferation was measured using CellTiter 96 AQueous with MTS reagent (Promega, Madison, Wis.). Briefly, cells in RPMI-medium plus 10% fetal bovine serum were added to 96-well plates at 4,000 cells per well in quadruplicate. After 24 h, LY290042, Ku0063794, Rapamycin, CCI-779 or BEZ-235 alone or combined were added, and cells were cultured for the indicated time. DMSO was used as a control. At 24 h post siRNA transfection, cells were treated with DMSO control or CCI-779 and incubated up to 72 h. MTS and phenazine methosulfate solution (20 μL/well) was added and the absorbance at 490 nm was recorded using a microplate reader (BMG Labtech; Cary, N.C.). For the clonogenic assay, 500 cells per well were seeded and cultured in 6-well plates for a week in the presence of Dox (0.5 μg/mL) in serum-fed conditions. The medium was changed every 3 days. Colonies were fixed with formaldehyde (4% v/v) and stained with crystal violet (0.5%). Representative views from triplicate experiments were photographed and quantified. For the soft agar colony formation assay, 5×103 cells were suspended in 1 ml of 0.3% agarose with Dox (0.5 μg/ml) and overlaid onto 1 ml of 0.5% solidified bottom agarose per well in 6-well plate. After solidification the top agarose was covered with 1 ml of RPMI with 10% FBS and Dox (0.5 μg/ml). The culture medium was changed every 3 days. After 14 days, colonies were photographed and quantified. For the sphere-forming assay in Matrigel, 80 μL Matrigel was added per well in eight-well chamber slides. After 30 min, 500 to 1000 cells per well suspended in 400 μL ice-cold 10%

Matrigel in phenol red-free RPMI medium. Cells were overlaid with 200 μL RPMI 10% FBS with Dox (0.5 μg/ml) and grown for 10 to 14 days with a change of medium every 3 days. Spheres were photographed and manually quantified. Xenograft experiments were conducted as previously described (11) and according to the protocol approved by the Institutional Animal Care and Use Committee.

### Example 5

### Cell Transfections and Reporter Assays

LNCaP or C4-2 cells were cultured in T-Medium with 5% fetal bovine serum (FBS; Gemini Bio Products; West Sacramento, Calif.) or in RPMI 1640 with 10% FBS, and HEK 293T cells were cultured in high glucose DMEM medium (Invitrogen, Inc.) with 10% FBS. All media were supplemented with 1% penicillin and streptomycin (Pen/Strep). Cells were incubated at 37° C. supplemented with 5% CO₂. Small interfering RNA (siRNA) specific to Mst1 and scrambled (control) siRNA were purchased from Thermo Scientific/Dharmacon RNAi Technologies (Roxford, Ill.). Double-stranded oligonucleotides (siRNAs) were transfected using DharmaFECT-2 transfection reagent (Thermo Scientific). Plasmid transfections with Lipofectamine 2000 were conducted according to the manufacturer's instructions. Luciferase reporter gene activity was determined using a Luciferase Assay System from Promega (Madison, Wis.) and a bioluminescence microplate reader (BMG Labtech). Relative Light Units (RLUs) were normalized to total protein and the data was presented as luciferase activity. The tetracycline-inducible C4-2 cell model with stable HA-tagged Mst1-T120A expression was established as described previously (11) according to the manufacturer's instructions (Clontech Laboratories, Inc., Mountain View, Calif.).

### Example 6

### Imaging and Microscopy

Cells were seeded on sterile 8-well chamber slides at 70% confluence and fixed in 3% paraformaldehyde for 30 min at room temperature for blocking and antibody labeling. Probe included Alexa Flour® 488 conjugated with secondary goat anti-rabbit antibody (1:500). Cell nuclei were detected by DAPI staining (Vector Laboratory, Burlingame, Calif.). Cells were imaged at 20× magnification by fluorescence microscopy (Nikon Eclipse Ti model; USA). Immunohistochemistry (IHC) was performed on 5-micron thick paraffin sections. Tissue slides were de-paraffinized and rehydrated using standard techniques. Antigen retrievals were achieved by 5 min pressure-cooking and then cooling down to room temperature for 1 h. Blocking was performed by double endogenous enzyme block in 10 min. Tissues were incubated with primary antibodies (phospho-Mst1-T120 and phospho-Mst1-T183) at 4° C. overnight. They were subjected to DakoCytomation EnVision plus horseradish peroxidase reagent for 30 min. Signals were detected by adding substrate hydrogen peroxide using diaminobenzidine as chromogen and counterstained by hematoxylin. Slides were then dehydrated and mounted. All reagents were obtained from Dako Corporation (Carpinteria, Calif.). All experiments with human subjects were conducted according to a protocol approved by the Institutional Review Board.

### Example 7

### Statistical Analysis

Values are expressed as mean ±SD. An unpaired t-test was conducted to analyze for differences between treatments. Statistical significance was set at p≦0.05.

### Example 8

### Mst1-T120 Phosphorylation is Enriched in Prostate Cancer Cell Nuclei

The inventors sought to investigate Mst1-T120 phosphorylation to gain insight into the mechanisms regulating Mst1 in prostate cancer cells since the loss or reduction of Mst1 function has been implicated in prostate cancer progression to the castration-resistant cell phenotype in humans (10, 11). The T120 residue is a potential Akt (Akt1) phosphorylation signature and T120 phosphorylation by Akt has been suggested to prevent Mst1 activation (8) by preventing caspase cleavage and nuclear localization in ovarian cancer cells (6). In this study, LNCaP and its castration-resistant C4-2 cell subline were used because these cell models are androgen receptor (AR) positive and possess hyperactivation of PI3K/Akt/mTOR signaling, which is central to prostate cancer cell survival and metastasis (20, 21).

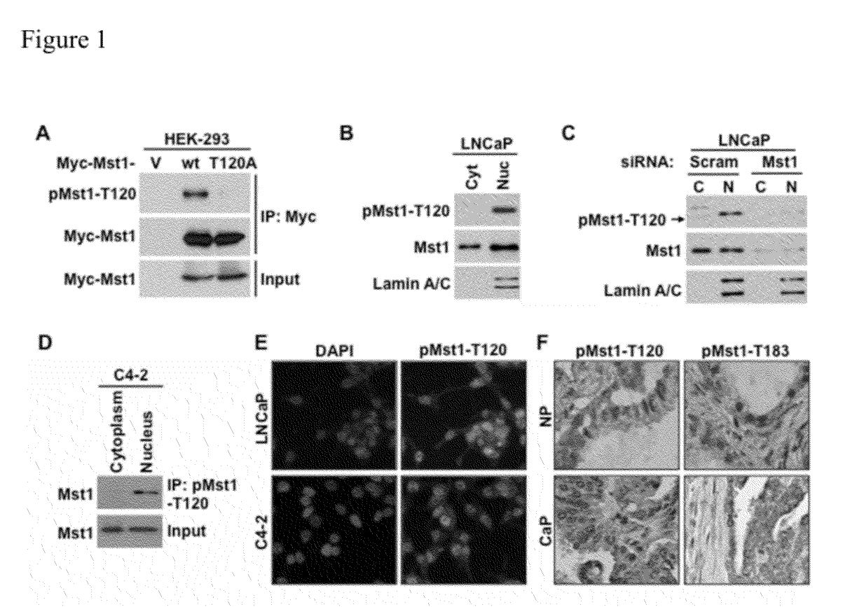

The inventors generated a custom-designed and site-specific rabbit polyclonal antibody using the chemically synthesized and T120-phosphorylated peptide corresponding to the NH2-terminus of human Mst1 to carry out this investigation. The inventors verified the specificity of the antibody against phospho-Mst1-T120 protein using Myc-tagged phosphorylation-deficient (T120A) Mst1 mutant, Mst1-wt or vector control expressed in HEK 293 cells. As revealed by immunoprecipitation (IP) and western blot analysis, neither vector nor phosphorylation-deficient Mst1-T120A mutants showed any reactivity with phospho-T120 peptide antibody compared to Mst1-wt (FIG. 1A), indicating that the antibody is specific to phospho-Mst1-T120 protein.

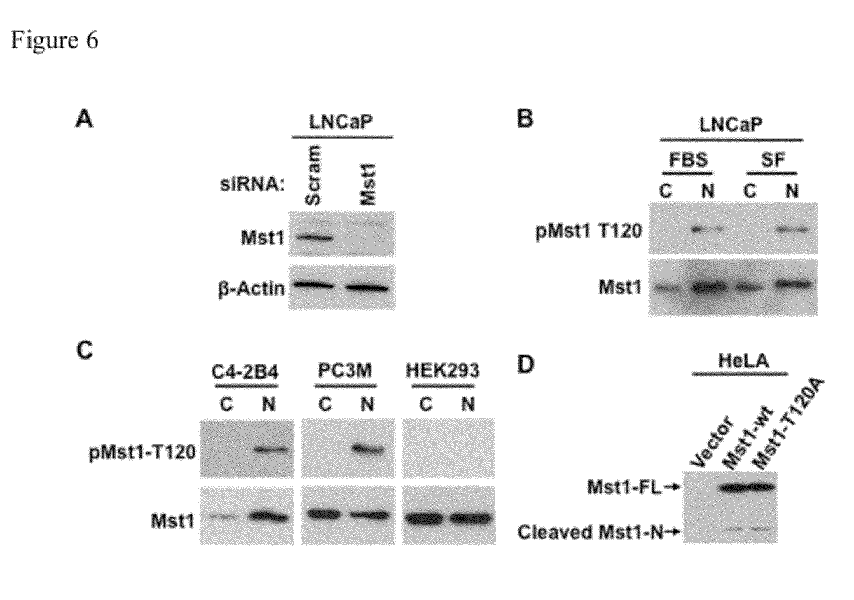

The inventors' studies and those of others have shown that Mst1 localizes in cytoplasm and nucleus (8, 10). To determine the site where phospho-Mst1-T120 phosphorylation is enriched in the cell, the levels of phospho-Mst1-T120 were assessed by western blot in cytoplasmic and nuclear fractions isolated from LNCaP cells. As shown in FIG. 1B and FIG. 1C, Mst1-T120 phosphorylation was almost exclusively enriched in nuclear fractions. In addition, RNAi knockdown (FIG. 1C and FIG. 6A) and immunoprecipitation (IP) (FIG. 1D) experiments further confirmed the above data showing that the antibody specifically recognized phospho-Mst1-T120 protein in the nucleus. As revealed by immunofluorescence (IF) experiments, a similar distribution of Mst1-T120 phosphorylation was observed in LNCaP and C4-2 cells in serum-fed conditions (FIG. 1E). Serum starvation did not significantly alter the nuclear enrichment of Mst1-T120 phosphorylation (FIG. 6B). A similar distribution of phospho-Mst1-T120 protein was also observed in C4-2B4, a bone metastatic subline of C4-2 cells or in PC3M cells (FIG. 6C). Mst1-T120 phosphorylation appears to be specific to cancerous cells because no detectable Mst1-T120 phosphorylation was recorded in non-transformed cells such as HEK 293 (FIG. 6C).

The inventors then examined the levels of phospho-Mst1-T120 in normal and cancerous prostate tissues by immunohistochemistry (IHC). The results of this experiment showed a similar pattern of phospho-T120 distribution in clinical samples, and the number of cells reacted with the phospho-T120 antibody and the signal intensity were dramatically increased in cancerous tissue compared to the non-cancerous counterpart (FIG. 1F). The inventors also examined the levels of Mst1 -T183 phosphorylation, another important site for protein kinase activity and apoptotic function, in prostate tissue samples and showed that T183 phosphorylation was detected only in the cytoplasm, but not in cell nuclei, with increasing levels in cancer tissues (FIG. 1F).

### Example 9

### Mst1-T120 Phosphorylation is Not a Direct Physiologic Target of PI3K-Akt Signaling in Prostate Cancer Cell Nuclei

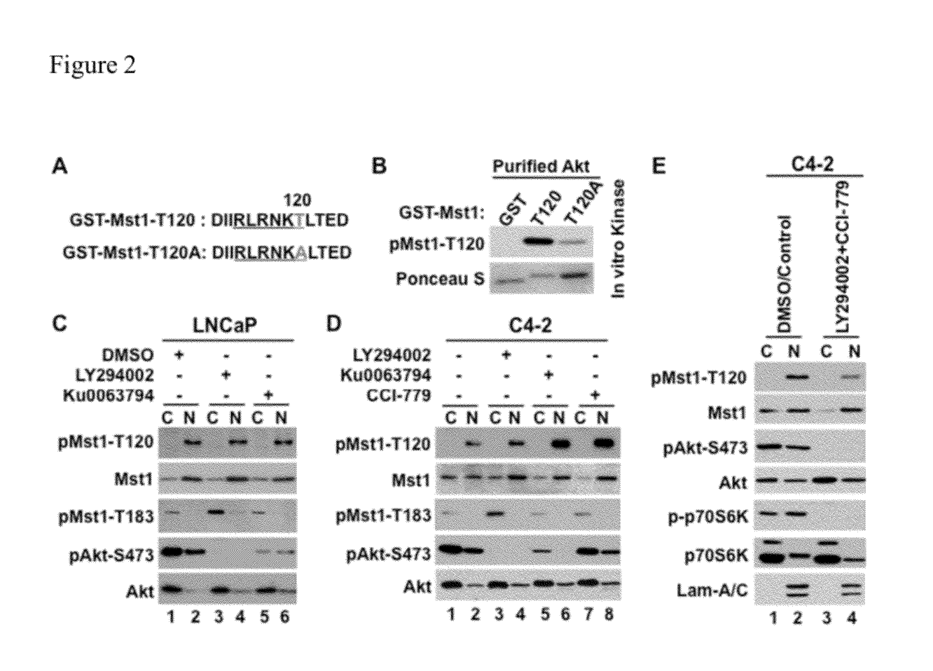

The inventors generated GST-Mst1 peptide with T120-wt or T120A mutations (FIG. 2A). Non-radioactive kinase assays with the pre-activated recombinant Akt showed that Akt phosphorylates the purified GST-Mst1- T120 peptide in vitro and the phosphorylation is specific because the kinase assay with GST-only or GST-Mst1-T120A mutant peptide displayed an undetectable or very low signal, respectively (FIG. 2B). The inventors then assessed the effects of PI3K-Akt inhibition on Mst1-T120 phosphorylation in its respective location in the cell. LNCaP cells were treated with vehicle (DMSO or control) or a selective pharmacological PI3K inhibitor, LY294002, which chemically inhibits Akt activity (22, 23). The levels of phospho-Mst1-T120 were assessed by western blot in cytoplasmic and nuclear fractions. PI3K/Akt inhibition did not significantly affect Mst1-T120 phosphorylation in cell nuclei in comparison to the control, even though Akt activity was completely abolished (FIG. 2C, lane 4 vs. lane 2). A similar observation was also made in C4-2 cells in response to LY294002 under the same experimental conditions (FIG. 2D, lane 3 vs. lane 1). While not wishing to be bound by any one particular theory, these findings suggest that Mst1-T120 phosphorylation is not a direct physiologic target of PI3K-Akt signaling in vivo.

### Example 10

### Mst1-T120 Phosphorylation is Enhanced by mTOR Inhibition in Prostate Cancer Cells

The serine-threonine kinase mTOR is an important downstream mediator of PI3K/Akt signaling (24). mTOR exists in two protein complexes (25): a rapamycin sensitive mTOR complex 1 (mTORC1) and a rapamycin insensitive mTOR complex 2 (mTORC2). The inventors wanted to determine whether mTOR signaling would regulate Mst1-T120 phosphorylation, and if so, which of the mTOR complexes contributes to this event. LNCaP or C4-2 cells were treated with Ku0063794, a potent mTORC1/C2inhibitor, or CCI-779, a potent mTORC1 inhibitor. FIG. 2C and FIG. 2D show that Ku0063794 was unable to prevent Mst1-T120 phosphorylation in cell nuclei of LNCaP cells (lane 6 vs. lane 2), in comparison to the control (DMSO). Nevertheless, Ku0063794 or CCI-779 resulted in Mst1-T120 hyperphosphorylation and caused an increase in Mst1 nuclear accumulation in C4-2 cells (FIG. 2D). Combinatorial PI3K and mTORC1 inhibition synergistically reduced Mst1-T120 phosphorylation in C4-2 cells in comparison to the control (FIG. 2E, lane 4 vs. lane 2) or single agent (FIG. 2D, lane 8 vs. lane 2).

### Example 11

### Mst1-T120 Phosphorylation Confers Resistance to mTOR Inhibition in Castration-Resistant Prostate Cancer Cells

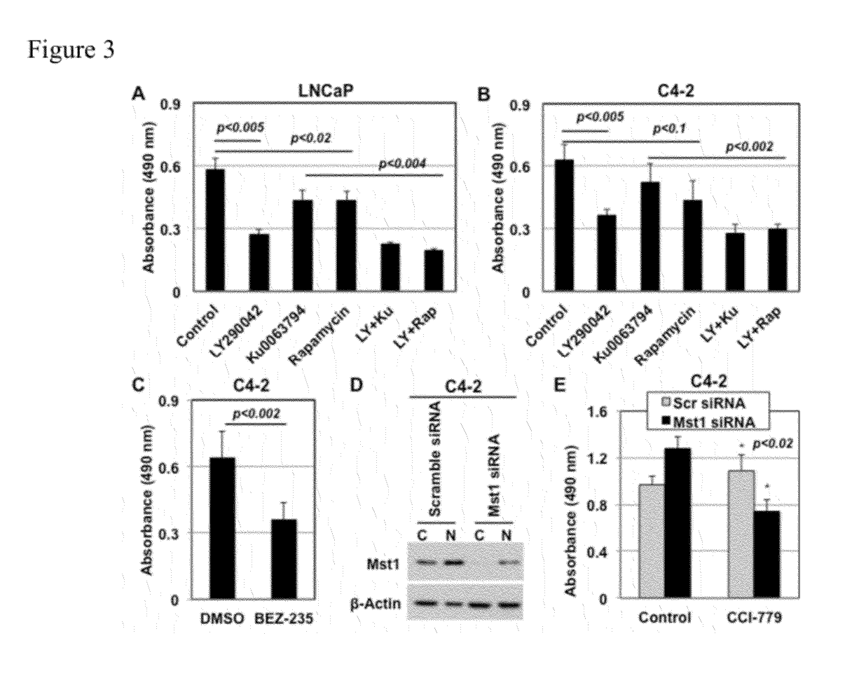

To test whether sustained Mst1-T120 phosphorylation is associated with resistance to growth reduction by mTOR inhibition, LNCaP or C4-2 cells were treated with DMSO, LY294002, Ku0063794, rapamycin alone, LY294002 plus Ku0063794, or LY294002 plus rapamycin. As shown in FIG. 3A, combinatorial PI3K and mTOR inhibition had significantly better growth inhibitory effects (p<0.004) than the single agent (p<0.02) in LNCaP cells. However, Ku0063794 or rapamycin as a single agent failed to significantly inhibit C4-2 cell growth in comparison to the control (p<0.1) (FIG. 3B). Consistent with the reduction of Mst1-T120 phosphorylation, co-administration of LY294002 plus Ku0063794 or LY294002 plus rapamycin significantly reduced the growth of C4-2 cells in comparison to treatment with a single agent (p<0.002) (FIG. 3B). In addition, a dual PI3K-mTOR inhibitor, BEZ-235, significantly inhibited C4-2 cell proliferation (p<0.002) (FIG. 3C).

The inventors then performed a growth assay in the presence and absence of Mst1 knockdown conditions, which would reduce T120 phosphorylation, to assess whether aberrant Mst1 signaling due to T120 hyper-phosphorylation is directly associated with resistance to mTORC1 inhibition by CCI-779. The growth assay had to be conducted in Mst1-knockdown conditions because of the unavailability of Mst1 pharmacologic inhibitor. The data in FIG. 3D and FIG. 3E demonstrated that Mst1 knockdown sensitized C4-2 cells to mTOR inhibition by CCI-779 compared to the scramble siRNA control. As expected, Mst1 knockdown in control conditions resulted in growth acceleration because Mst1 itself functions as a growth suppressor (11). While not wishing to be bound by any one particular theory, these findings support the conclusion that the deregulation of Mst1, possibly as a result of sustained T120 phosphorylation, is associated with resistance to mTOR inhibition.

### Example 12

### Mst1-T120 Phosphorylation Limits the Ability of Mst1 to Restrict Cell Proliferation in Prostate Cancer Cells

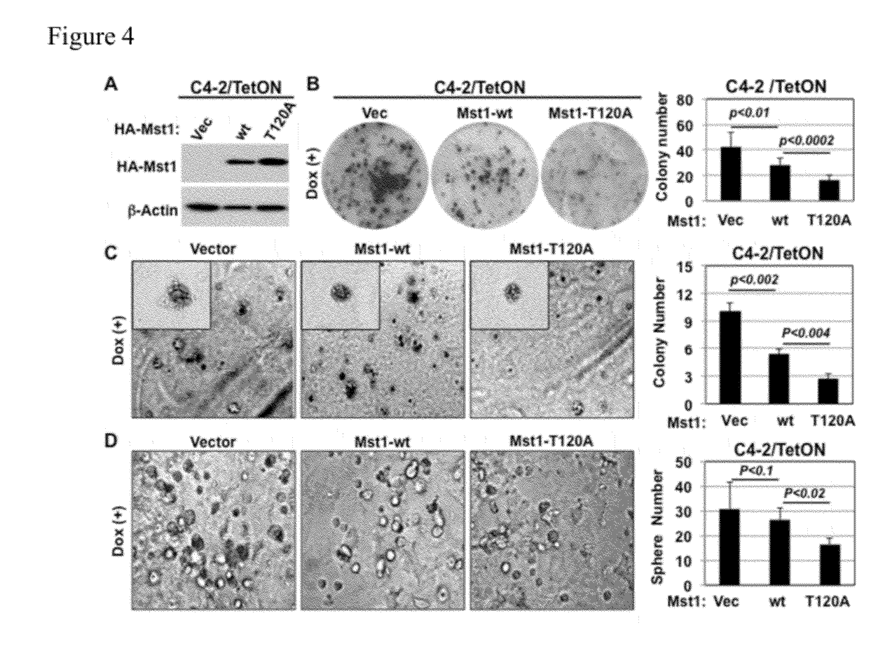

To assess the effects of T120 phosphorylation on the Mst1 mediation of growth restriction, the inventors engineered C4-2 cells to express stable and HA tagged phosphorylation-deficient Mst1 mutant protein (Mst1-T120A) under the control of tetracycline or doxycycline (Dox) inducible promoter (FIG. 4A). The inventors performed a series of biological assays in cultures using C4-2/Mst1-T120A along with C4-2/vector (negative control) or C4-2/Mst1-wt (positive control) cells, which were developed earlier (11). First, enforced Mst1-T120A mutant expression prevented the clonogenic ability of C4-2 cells in vitro compared to the Mst1-wt or vector control (FIG. 4B). Second, the induction of mutant Mst1-T120A protein in C4-2 cells significantly reduced the number and size of colonies formed in soft agar in comparison to the Mst1-wt or vector control (p<0.004) (FIG. 4C). Third, enforced Mst1-T120A mutant expression in C4-2 cells resulted in the formation of significantly fewer spheres in Matrigel than the Mst1-wt or vector control (p<0.02) (FIG. 4D).

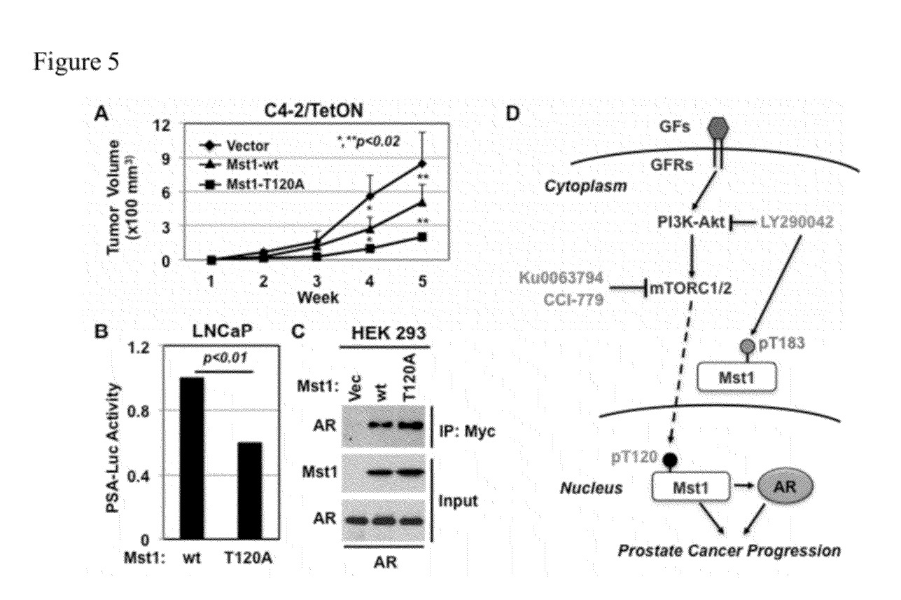

The inventors then performed a xenograft experiment to test whether the induction of Mst1-T120A mutant protein would alter the tumor-forming ability of C4-2 cells in vivo through interaction with skin fibroblasts. Immunocompromised and nude male mice were inoculated with inducible C4-2/vector, C4-2/HA-Mst1-wt, or C4-2/HA-Mst1-T120A cells subcutaneously, and animals were then treated with Dox (0.5 mg/ml) in drinking water for six weeks. Tumor sizes were measured manually every week. Consistent with in vitro data, C4-2/Mst1-T120A cells produced smaller tumors than Mst1-wt or vector control (FIG. 5A).

### Example 13

### Mst1-T120 Phosphorylation Restricts the Ability of Mst1 to Antagonize AR-Dependent Gene Expression in Prostate Cancer Cells

A previous study from the inventors' laboratory suggested that Mst1 antagonized AR-dependent gene expression in prostate cancer cells (11). This finding was leveraged to assess whether T120 phosphorylation alters the effects of Mst1 on AR, given that Mst1-T120 phosphorylation is primarily enriched in cell nuclei. The inventors conducted a prostate specific antigen (PSA) promoter-luciferase reporter (PSA-Luc) assay, which is a well-characterized AR-regulated promoter in prostate cancer cells (26). The data in FIG. 5B showed that enforced Mst1-T120A expression inhibited AR activation about 50% more (p<0.01) than Mst1-wt. The inventors did not observe any alteration in AR-dependent PSA promoter activation by the induction of Mst1-T387A mutant protein, another potent Akt phosphorylation site in Mst1, relative to the Mst1-wt (not shown).

The inventors reported that protein-protein interaction appears to play an important role in the suppression of AR-dependent gene expression by Mst1 (11). The inventors then determined whether Mst1-T120A mutant protein could form an enhanced protein complex with AR to have a greater inhibitory effect on AR activity than Mst1 -wt. Full-length AR was transiently co-expressed with vector, Mst1-wt or Mst-T120A in HEK-293 cells. As revealed by co-immunoprecipitation and western blot analysis, the protein-protein interaction between AR and Mst1-T120A mutant proteins was indeed about 50% greater than the interaction between the AR and Mst1-wt (FIG. 5C). These observations indicate that T120 phosphorylation restricts the Mst1 diminution of AR activity in prostate cancer cells.

### Example 14

### Antibodies and Reagents

Antibodies to NH₂-terminal Mst1 from Cell Signaling Technology (Danvers, Mass.) and to COOH-terminal Mst1 (STK4), from Novus Biologicals (Littleton, Colo.) and Abnova (Walnut, Calif.) were obtained. The antibodies to AR were from Millipore (Billerica, Mass.), to HA-tag from Covance (Berkeley, Calif.), to Myc-tag from BD Biosciences (Mountain View, Calif.), Lamin A/C from Cell Signaling Technology (Danvers, Mass.) and β-actin from Santa Cruz (Santa Cruz, Calif.). Phospho-antibody to Mst1-T183, Akt-S473, and p70S6K were purchased from Cell Signaling Technology. HRP-conjugated rabbit and mouse secondary antibody were obtained from Thermo Scientific/Pierce (Rockford, Ill.), or GE Health Care (Piscataway, N.J.). Alexa Fluor® 488 conjugated secondary antibody was obtained from Molecular Probes (Grand Island, N.Y.). Chemiluminescence reagent SuperSignal was from Thermo Scientific. Transfection reagents and Lipofectamine 2000 were from Invitrogen, Inc. (Grand Island, N.Y.) and Fugene6 was from Roche, (South San Francisco, Calif.). Doxycycline (Dox) was from Sigma-Aldrich (St. Louis, Mo.). Protein A- or Protein G- Sepharose, and GST-Sepharose were from GE Healthcare (Pasadena, Calif.). Specific inhibitors to PI3K (LY290042) were from Calbiochem (Philadelphia, Pa.), those to mTOR (Ku0063794) were from Chemdea (Ridgewood, N.J.), and those to rapamycin (sirolimus) and CCI-779 (temsirolimus) were from Sigma-Aldrich and the dual inhibitor (BEZ-235) to PI3K and mTOR was from Selleck Chemicals (Houston, Tex.).

### Example 15

### Discussion

In this study, the inventors described a new mechanism of Mst1 regulation by phosphorylation that likely associates with resistance to mTOR inhibitors in prostate cancer cells. Evidence supporting this conclusion includes: (i) the nuclear, but not the cytoplasmic, Mst1 is phosphorylated at the T120 residue, (ii) Mst1-T120 phosphorylation may not be a direct, but may be indirect, target of PI3K and mTOR signaling, (iii) hyper-phosphorylation of Mst1-T120 may promote resistance to mTOR inhibition, (iv) persistent T120 phosphorylation significantly limits the ability of Mst1 to restrict prostate cancer cell growth in vitro and tumor growth in xenografts and significantly reduced the Mst1 diminution of AR-dependent gene expression in prostate cancer cells. Collectively, these findings indicate that an altered regulation of Mst/hippo signaling by mTOR may have important biological consequences and therapeutic implications in prostate cancer.

Here, the inventors found that the inhibition of mTOR by Ku0063794 or CCI-779 resulted in Mst1-T120 hyper-phosphorylation in castration-resistant, but not in castration sensitive, prostate cancer cells (FIG. 2D, lanes 6 and 8). The inventors did not observe Akt activation under the same conditions above. Combinatorial PI3K and mTORC1 inhibition synergistically prevented Mst1-T120 phosphorylation about 50% below baseline. Consistent with this finding, PI3K and mTORC1 inhibition combined synergistically to prevent the proliferation of C4-2 cells compared to a single agent (FIG. 3B). Additional data suggested that Mst1 knockdown sensitized C4-2 cells to mTORC1 inhibition by CCI-779 (FIG. 3D, 3E). While not wishing to be bound by any one particular theory, these findings suggest the possibility that deregulated Mst1, particularly in advanced prostate cancer, promotes cell survival under conditions where Akt activity is inhibited. While not wishing to be bound by any one particular theory, this may be an important mechanism by which prostate cancer acquires resistance to mTOR inhibition as an alternative or in parallel to Akt activation.

Using the inventors own custom-designed and validated phospho-T120 peptide antibody, they found that nuclear, but not cytoplasmic, Mst1 protein was phosphorylated and that phosphorylation does not have a significant impact on Mst1 cleavage (FIG. 6D). The inventors also found that the majority of total and phospho-Mst1-T120 protein was accumulated in the cytoplasm in breast or ovarian cancer cells (not shown). Therefore, while not wishing to be bound by any one particular theory, it's likely that multiple Mst1 forms exist that can be regulated in a context-dependent or tissue-specific manner. This is likely to explain differences between the inventors' findings here and findings in the literature (6). Mst1 is a multifunctional caspase independent protein (11) and regulates diverse cellular functions (18). While not wishing to be bound by any one particular theory, it is likely that posttranslational modifications, such as by phosphorylation, could provide unique opportunities for Mst1 to accommodate such diverse functions. In addition, given that Mst1 can be found in multiple cell locations including lipid rafts, cytoplasm or nuclei, it remains to be determined whether Mst1 has a location-specific function in the cell.

Moreover, Mst1 phosphorylation at T120 was suggested to inhibit T183 phosphorylation in Mst1, which is another important regulatory mechanism for Mst1 activation and apoptosis (6, 19). Here the inventors demonstrated that Mst1-T183 phosphorylation was detected in the cytoplasm, but not in cell nuclei. Furthermore, the inventors' observations are consistent with published studies (6, 19) indicating that a potent PI3K inhibitor, LY294002, enhanced Mst1-T183 phosphorylation (FIG. 3). However, the levels of T183 phosphorylation were unchanged when cells were treated with a potent mTOR inhibitor, Ku0063794, (FIG. 3). The inventors' IHC data further demonstrated that T183 phosphorylation was increased in prostate tumor tissue samples compared to noncancerous counterparts (FIG. 1F). Based on these observations, the inventors therefore conclude that Mst1-phosphorylation at T120 or at T183 are unrelated events or is modulated by different mechanisms, at least in prostate cancer cells.

Furthermore, the inventors' data indicate that nuclear-Mst1 protein was constitutively phosphorylated at the T120 residue and that can be further enhanced by mTOR inhibition in castration-resistant C4-2 cells, but not in the castration-sensitive LNCaP parental line. The inventors' data also indicate that the induction of T120 phosphorylation correlates with C4-2 cell resistance to mTOR inhibition and may negatively regulate the Mst1 diminution of cell growth and AR dependent gene expression (11). The inventors previously published study revealed that the growth suppressive effects of Mst1 were significantly diminished in C4-2 cells compared to LNCaP, even though both cell lines expressed similar levels of exogenous Mst1 protein (11). Observations from this and previous studies support the idea that deregulation of Mst1 could play a significant role in prostate cancer progression and chemo-resistance.

In summary, the inventors have proposed a model shown in FIG. 5D by which mTORC1/2 signaling complexes downstream of the PI3K/Akt pathway may indirectly regulate Mst1-T120 phosphorylation in cell nuclei. While not wishing to be bound by any one particular theory, the mechanism herein described may shed new light on how prostate cancer evolves and acquires resistance to chemotherapy, particularly mTOR inhibition.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

1. Hay B A, Guo M. Coupling cell growth, proliferation, and death. Hippo weighs in. Dev Cell. 2003; 5:361-3.
2. Harvey K F, Pfleger C M, Hariharan I K. The Drosophila Mst ortholog, hippo, restricts growth and cell proliferation and promotes apoptosis. Cell. 2003; 114:457-67.
3. Creasy C L, Ambrose D M, Chernoff J. The Ste20-like protein kinase, Mst1, dimerizes and contains an inhibitory domain. J Biol Chem. 1996; 271:21049-53.
4. Praskova M, Khoklatchev A, Ortiz-Vega S, Avruch J. Regulation of the MST1 kinase by autophosphorylation, by the growth inhibitory proteins, RASSF1 and NORE1, and by Ras. Biochem J. 2004; 381:453-62.
5. Jang S W, Yang S J, Srinivasan S, Ye K. Akt phosphorylates Mst1 and prevents its proteolytic activation, blocking FOXO3 phosphorylation and nuclear translocation. The Journal of biological chemistry. 2007; 282:30836-44.
6. Yuan Z, Kim D, Shu S, Wu J, Guo J, Xiao L, et al. Phosphoinositide 3-kinase/Akt inhibits MST1-mediated proapoptotic signaling through phosphorylation of threonine-120. J Biol Chem. 2009.
7. Graves J D, Draves K E, Gotoh Y, Krebs E G, Clark E A. Both phosphorylation and caspase-mediated cleavage contribute to regulation of the Ste20-like protein kinase Mst1 during CD95/Fas-induced apoptosis. The Journal of biological chemistry. 2001; 276:14909-15.
8. Ura S, Masuyama N, Graves J D, Gotoh Y. Caspase cleavage of MST1 promotes nuclear translocation and chromatin condensation. Proc Natl Acad Sci U S A. 2001; 98:10148-53.
9. de Souza P M, Lindsay M A. Mammalian Sterile20-like kinase 1 and the regulation of apoptosis. Biochem Soc Trans. 2004; 32:485-8.
10. Cinar B, Fang P K, Lutchman M, Di Vizio D, Adam R M, Pavlova N, et al. The pro-apoptotic kinase Mst1 and its caspase cleavage products are direct inhibitors of Akt1. EMBO J. 2007; 26:4523-34.
11. Cinar B, Kisaayak Collak F, Lopez D, Akgul S, Mukhopadhyay N K, Kilicarslan M, et al. MST1 is a Multifunctional Caspase-Independent Inhibitor of Androgenic Signaling. Cancer research. 2011.
12. Steinmann K, Sandner A, Schagdarsurengin U, Dammann R H. Frequent promoter hypermethylation of tumor-related genes in head and neck squamous cell carcinoma. Oncol Rep. 2009; 22:1519-26.
13. Seidel C, Schagdarsurengin U, Blumke K, Wurl P, Pfeifer G P, Hauptmann S, et al. Frequent hypermethylation of MST1 and MST2 in soft tissue sarcoma. Mol Carcinog. 2007; 46:865-71.
14. Qiao M, Wang Y, Xu X, Lu J, Dong Y, Tao W, et al. Mst1 is an interacting protein that mediates PHLPPs' induced apoptosis. Mol Cell. 2010; 38:512-23.
15. Minoo P, Zlobec I, Baker K, Tornillo L, Terracciano L, Jass J R, et al. Prognostic significance of mammalian sterile20-like kinase 1 in colorectal cancer. Mod Pathol. 2007; 20:331-8.
16. Lu L, Li Y, Kim S M, Bossuyt W, Liu P, Qiu Q, et al. Hippo signaling is a potent in vivo growth and tumor suppressor pathway in the mammalian liver. Proc Natl Acad Sci U S A. 107:1437-42.
17. Song H, Mak K K, Topol L, Yun K, Hu J, Garrett L, et al. Mammalian Mst1 and Mst2 kinases play essential roles in organ size control and tumor suppression. Proc Natl Acad Sci U S A. 107:1431-6.
18. Zeng Q, Hong W. The emerging role of the hippo pathway in cell contact inhibition, organ size control, and cancer development in mammals. Cancer Cell. 2008; 13:188-92.
19. Kim D, Shu S, Coppola M D, Kaneko S, Yuan Z Q, Cheng J Q. Regulation of proapoptotic mammalian ste20-like kinase MST2by the IGF1-Akt pathway. PloS one. 2010; 5:e9616.
20. Carver B S, Chapinski C, Wongvipat J, Hieronymus H, Chen Y, Chandarlapaty S, et al. Reciprocal feedback regulation of PI3K and androgen receptor signaling in PTEN-deficient prostate cancer. Cancer cell. 2011; 19:575-86.
21. Mulholland D J, Tran L M, Li Y, Cai H, Morim A, Wang S, et al. Cell autonomous role of PTEN in regulating castration-resistant prostate cancer growth. Cancer cell. 2011; 19:792-804.
22. Cinar B, Mukhopadhyay N K, Meng G, Freeman M R. Phosphoinositide 3-kinase-independent nongenomic signals transit from the androgen receptor to Akt1 in membrane raft microdomains. J Biol Chem. 2007; 282:29584-93.
23. Roper J, Richardson M P, Wang W V, Richard L G, Chen W, Coffee E M, et al. The dual PI3K/mTOR inhibitor NVP-BEZ235 induces tumor regression in a genetically engineered mouse model of PIK3CA wild-type colorectal cancer. PloS one. 2011; 6:e25132.
24. Ikenoue T, Hong S, Inoki K. Monitoring mammalian target of rapamycin (mTOR) activity. Methods in enzymology. 2009; 452:165-80.
25. Zhou H, Luo Y, Huang S. Updates of mTOR inhibitors. Anticancer Agents Med Chem. 2010; 10:571-81.
26. Cleutjens K B, van der Korput H A, van Eekelen C C, van Rooij H C, Faber P W, Trapman J. An androgen response element in a far upstream enhancer region is essential for high, androgen-regulated activity of the prostate-specific antigen promoter. Mol Endocrinol. 1997; 11:148-61.
27. Deocampo N D, Huang H, Tindall D J. The role of PTEN in the progression and survival of prostate cancer. Minerva Endocrinol. 2003; 28:145-53.
28. Zoncu R, Efeyan A, Sabatini D M. mTOR: from growth signal integration to cancer, diabetes and ageing. Nat Rev Mol Cell Biol. 2011; 12:21-35.
29. Sabatini D M. mTOR and cancer: insights into a complex relationship. Nat Rev Cancer.
30. Liu P, Cheng H, Roberts T M, Zhao J J. Targeting the phosphoinositide 3-kinase pathway in cancer. Nat Rev Drug Discov. 2009; 8:627-44.
31. Liu Q, Thoreen C, Wang J, Sabatini D, Gray N S. mTOR Mediated Anti-Cancer Drug Discovery. Drug Discov Today Ther Strateg. 2009; 6:47-55.
32. Morgan T M, Koreckij T D, Corey E. Targeted therapy for advanced prostate cancer: inhibition of the PI3K/Akt/mTOR pathway. Curr Cancer Drug Targets. 2009; 9:237-49.
33. Zeng Z, Sarbassov dos D, Samudio I J, Yee K W, Munsell M F, Ellen Jackson C, et al. Rapamycin derivatives reduce mTORC2 signaling and inhibit AKT activation in AML. Blood. 2007; 109:3509-12.
34. Behbakht K, Sill M W, Darcy K M, Rubin S C, Mannel R S, Waggoner S, et al. Phase II trial of the mTOR inhibitor, temsirolimus and evaluation of circulating tumor cells and tumor biomarkers in persistent and recurrent epithelial ovarian and primary peritoneal malignancies: a Gynecologic Oncology Group study. Gynecol Oncol. 2011; 123:19-26.
35. Bhagwat S V, Crew A P. Novel inhibitors of mTORC1 and mTORC2. Curr Opin Investig Drugs. 2010; 11:638-45.
36. Meric-Bernstam F, Gonzalez-Angulo A M. Targeting the mTOR signaling network for cancer therapy. Journal of clinical oncology : official journal of the American Society of Clinical Oncology. 2009; 27:2278-87.
37. Tamburini J, Chapuis N, Bardet V, Park S, Sujobert P, Willems L, et al. Mammalian target of rapamycin (mTOR) inhibition activates phosphatidylinositol 3-kinase/Akt by up-regulating insulin-like growth factor-1 receptor signaling in acute myeloid leukemia: rationale for therapeutic inhibition of both pathways. Blood. 2008; 111:379-82.
38. Fayard E, Xue G, Parcellier A, Bozulic L, Hemmings B A. Protein kinase B (PKB/Akt), a key mediator of the PI3K signaling pathway. Curr Top Microbiol Immunol. 2010; 346:31-56.
39. Ding Z, Liang J, Li J, Lu Y, Ariyaratna V, Lu Z, et al. Physical association of PDK1 with AKT1 is sufficient for pathway activation independent of membrane localization and phosphatidylinositol 3 kinase. PloS one. 2010; 5:e9910.
40. Werzowa J, Cejka D, Fuereder T, Dekrout B, Thallinger C, Pehamberger H, et al. Suppression of mTOR complex 2-dependent AKT phosphorylation in melanoma cells by combined treatment with rapamycin and LY294002. Br J Dermatol. 2009; 160:955-64.
41. Yang S, Xiao X, Meng X, Leslie K K. A Mechanism for Synergy with Combined mTOR and PI3 Kinase Inhibitors. PloS one. 2011; 6:e26343.
42. Guo C, Tommasi S, Liu L, Yee J K, Dammann R, Pfeifer G P. RASSF1A is part of a complex similar to the Drosophila Hippo/Salvador/Lats tumor-suppressor network. Curr Biol. 2007; 17:700-5.
43. Ghosh P M, Malik S N, Bedolla R G, Wang Y, Mikhailova M, Prihoda T J, et al. Signal transduction pathways in androgen-dependent and -independent prostate cancer cell proliferation. Endocr Relat Cancer. 2005; 12:119-34. What is claimed is:

1. A method of preventing cancer in an individual, comprising:
providing one or more compositions that directly or indirectly inhibit mTOR activity and PI3K activity; and
administering a therapeutically effective amount of one or more of the compositions to the individual so as to prevent cancer in the individual.

2. A method of inhibiting cancer in an individual, comprising:
providing one or more compositions that directly or indirectly inhibit mTOR activity and PI3K activity; and
administering a therapeutically effective amount of one or more of the compositions to the individual so as to inhibit cancer in the individual.

3. A method of treating cancer in an individual, comprising:
providing one or more compositions that directly or indirectly inhibit mTOR activity and PI3K activity; and
administering a therapeutically effective amount of one or more of the compositions to the individual so as to treat cancer in the individual.

4. A method of reducing a rate of cancer tumor development and/or progression to a metastatic state in an individual, comprising:
providing one or more compositions that directly or indirectly inhibit mTOR activity and PI3K activity; and
administering a therapeutically effective amount of one or more of the compositions to the individual, so as to reduce the rate of cancer tumor development and/or progression to the metastatic state in the individual.

5. The method of claim 1, wherein one or more of the compositions reduces a level of Mst1-T120 phosphorylation in an individual with cancer.

6. The method of claim 2, wherein one or more of the compositions reduces a level of Mst1-T120 phosphorylation in an individual with cancer.

7. The method of claim 3, wherein one or more of the compositions reduces a level of Mst1-T120 phosphorylation in an individual with cancer.

8. The method of claim 4, wherein one or more of the compositions reduces a level of Mst1-T120 phosphorylation in an individual with cancer.

9. The method of claim 1, wherein one or more of the compositions comprises LY294002 and/or Ku0063794.

10. The method of claim 2, wherein one or more of the compositions comprises LY294002 and/or Ku0063794.

11. The method of claim 3, wherein one or more of the compositions comprises LY294002 and/or Ku0063794.

12. The method of claim 4, wherein one or more of the compositions comprises LY294002 and/or Ku0063794.

13. The method of claim 1, wherein one or more of the compositions comprises BEZ-235.

14. The method of claim 2, wherein one or more of the compositions comprises BEZ-235.

15. The method of claim 3, wherein one or more of the compositions comprises BEZ-235.

16. The method of claim 4, wherein one or more of the compositions comprises BEZ-235.

17. The method of claim 1, wherein one or more of the compositions comprises rapamycin and/or rapalogs.

18. The method of claim 2, wherein one or more of the compositions comprises rapamycin and/or rapalogs.

19. The method of claim 3, wherein one or more of the compositions comprises rapamycin and/or rapalogs.

20. The method of claim 4, wherein one or more of the compositions comprises rapamycin and/or rapalogs.

21. The method of claim 1, wherein the cancer is prostate cancer.

22. The method of claim 2, wherein the cancer is prostate cancer.

23. The method of claim 3, wherein the cancer is prostate cancer.

24. The method of claim 4, wherein the cancer is prostate cancer.

25. The method of claim 1, wherein the cancer is hormone refractory metastatic prostate cancer.

26. The method of claim 2, wherein the cancer is hormone refractory metastatic prostate cancer.

27. The method of claim 3, wherein the cancer is hormone refractory metastatic prostate cancer.

28. The method of claim 4, wherein the cancer is hormone refractory metastatic prostate cancer.

29. A kit for treating, inhibiting or preventing a cancer in a subject in need thereof, comprising:
(i) providing one or more compositions that directly or indirectly inhibit mTOR activity and PI3K activity; and
(ii) instructions for the use of the one or more compositions for treating, preventing or inhibiting the cancer in the individual.

30. The kit of claim 29, wherein the cancer is prostate cancer.

31. The kit of claim 29, wherein one or more of the compositions reduces a level of Mst1-T120 phosphorylation when administered to an individual with cancer.

32. The kit of claim 29, wherein one or more of the compositions comprises LY294002 and/or Ku0063794.

33. The kit of claim 29, wherein one or more of the compositions comprises BEZ-235.
